(19) United States
(12) Patent Application Publication
(10) Pub. No.: US 2007/0276192 A1
(43) Pub. Date: Nov. 29, 2007

(54) Method and Device for Evaluation of Bio-Psycho-Physical Influence of Radio, Television and Media Products Upon Humans

(76) Inventors: Jivko Jelev, Sofia (BG); Anton Antonov, Sofia (BG); Tatyana Galabova, Sofia (BG)

Correspondence Address: LADAS & PARRY, 26 WEST 61ST STREET, NEW YORK, NY, 10023, US

(21) Appl. No.: 10/552,637

(22) PCT Filed: Apr. 9, 2004
(86) PCT No.: PCT/BG04/00007

§ 371 Date: May 18, 2007

(30) Foreign Application Priority Data
Apr. 9, 2003 (BG) 107715

(51) Int. Cl. A61B 5/00

(52) U.S. Cl. 600/300

(57) ABSTRACT

Method and equipment for estimation the biopsychophysical influence of radio, TV and media product on the man, characterizing itself that the media product (1) acts on the person (2), holding in his hands a bottle with water (3), drop from which are taken and placed in a equipment measuring the surface tension and fluctuations the drop's surface potential (4) and in an other equipment measuring the water intramolecula energy spectrum (5) and the data from both systems together with data from equipment's determining the biophysical parameters of the person are processed by the computer (7) compatible with the data (8) from previous measurements of that person.

### FIELD OF THE INVENTION

The invention relates to the Method and equipment for estimation the biopsychophysical influence of radio, TV and media products on the man in stanteneonsiy and long term health

### BACKGROUND OF THE INVENTION

Statistical methods for investigation and application the positive action of music and pictures on the human health (therapy by music and pictures) are well known but, they don't possess equipment with reverse bond for measurement the exact grade of influence based on some physical measurable parameters

### DISCLOSURE OF THE INVENTION

The aim of the invention is to create a method and equipment for estimation the biopsychophysical influence of radio, TV and media products on the man, which can show the grade of their action on the human instantaneously and long term health.

The problem is solved by a method and two equipment for estimation the biopsychophysical influence of radio, TV and media products on the man. The method for estimation the bio-psychophysical influence of radio, TV and media products is characterized itself, that the person under the influence of radio, TV and media products holds in his hands a bottle with water and a measurements of the latter are carried out by two system, the dates from which together with the dates from equipment measuring the biophysical parameters of the person are processed by a computer compatible with the dates from previous measurements of that person. The first system for estimation the influence of radio, TV and media products on the man is characterized it self, that a drop from the water influenced by media product via the person under investigation is bid in a hermetic box with air pump on a transparent lamella which consist of a thin mailer folio and a glass plate, illuminated through a glass window with a plane parallel beam, forming on the bottom surface of the plate dark, ring, which is registered by the help of a mirror and a digital TV camera and by a computer the width of the dark ring and the energy distribution spectrum is calculated. The second for estimation the influence of radio, TV and media products on the man is characterized it self, that a water drop is laid in a box with air pump on a pad placed under the influence of radio, TV and media product through a hole in the box and with a reader, directed by a manipulator the surface tension and the fluctuations of the surface potential are measured and recorded in a computer.

### BRIEF DESCRIPTION OF THE DRAWINGS

FIG. 1 shows a block scheme of the method for estimation the bio-psychophysical influence of radio, TV and media products on the man
1. Influencing product (video, sound, pictures and others)
2. Man
3. Bottle with water holder in man's hands
4. Equipment, measuring the surface tension and fluctuations of the surface potential the water drop
5. Equipment, measuring the water intramolecular energy spectrum
6. Equipment, measuring the biophysical parameters of the man: 02 in the blood pressure, EKG and arithmy, EEG and others
7. Computer processing of the dates
8. Basic dates of previous measurements of the person

FIG. 2 shows equipment for estimation the influence of radio, TV and media products of the man
1. Source of a plane parallel beam
2. Glass window
3. Hermetic box
4. Water drop
5. Folio of mylar
6. Glass plate
7. Digital TV camera
8. Computer
9. Air pump
10. Mirror

FIG. 3 shows equipment for estimation the influence of radio, TV and media products on the man's

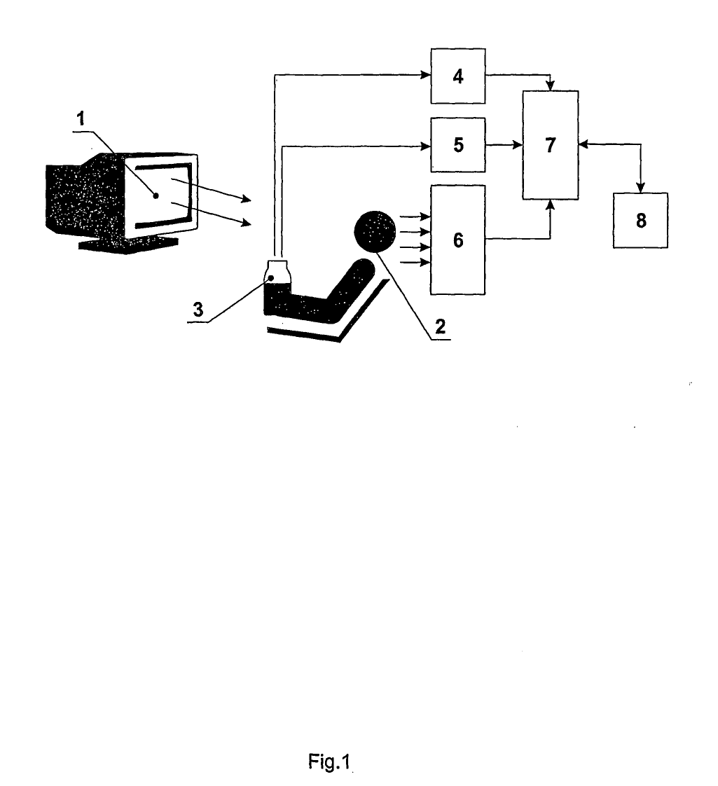

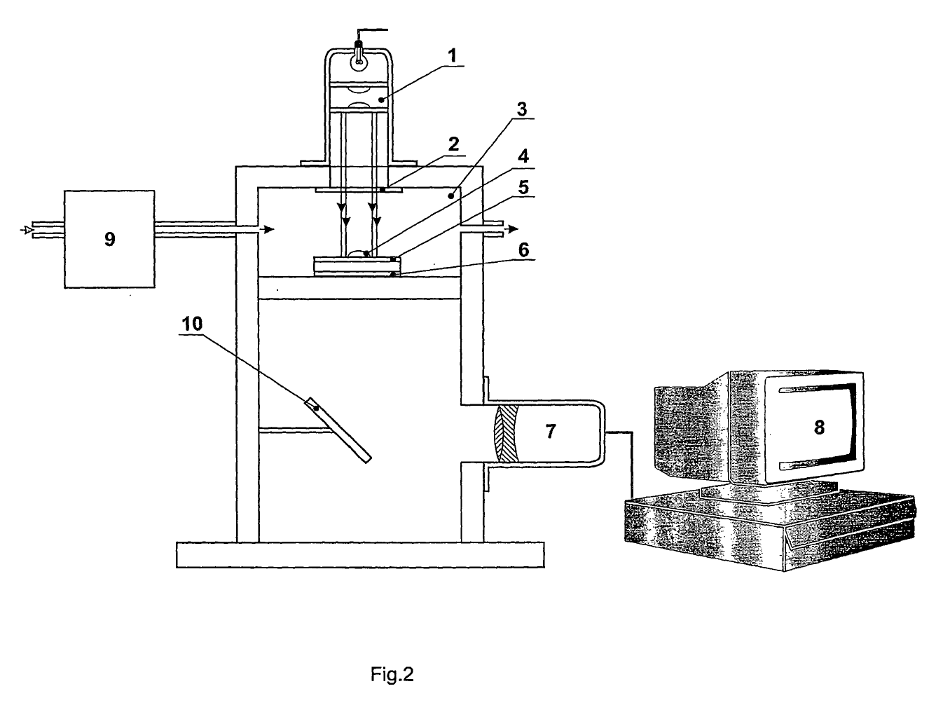

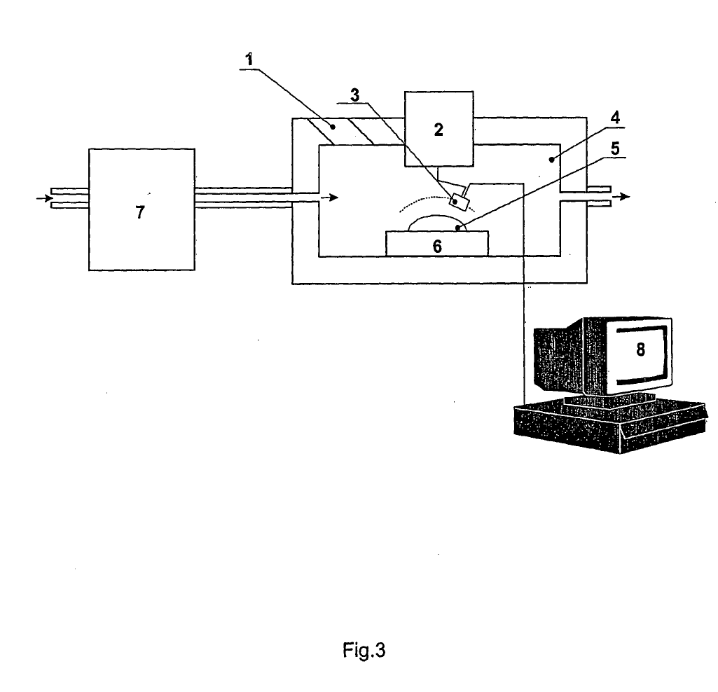

Hole in the box for a influence of radio, TV and other products
1. Manipulator
2. Reader
3. Work camera
4. Water drop
5. Pad
6. Air pump
7. Computer

### DESCRIPTION OF THE PREFERRED EMBODIMENTS

The equipment for estimation on the biopsychophysical influence of radio, TV and media products on the man, measuring the water intramolecular energy spectrum is realized by the following method:

The water intramolecular energy spectrum is determined by a measurement the wetting angles changes of water drops from treated and nontreated (control water), evaporating simultaneously at constant condition—temperature, relative humidity and air capacity. The energy of the H-bongs between two water molecules E is nounded with the wetting angle by the relation:

$$E = - \frac{1 + cos θ}{14 \text{,} 33}$$

The water intramolecular energy spectrum is defined by the expression:

$$f ( E ) = \frac{14 \text{,} 33 f ( θ )}{\sqrt{1 - {( 1 + 14 \text{,} 33 E )}^{2}}} ;$$

where f(θ) is the distribution function on wetting angles. It can received the latten by measurements at equal intervals of time the wetting angles θ until the fully evaporation of the water drops, which is realized at θ=0

The wetting angle can be determined by the following way: the water drops are laid on a hydrophobic transparent pad, which consist of a thin mylar folio and a glass plate. After illumination the drop with plane parallel beam and refraction of the light in its ends a dark ring has been received on the bottom surface of the plate. Its widths is connected with the wetting angle of the drop in the following manner:

$$θ = arc tg \frac{{n}_{1}}{{[ {n}_{2}^{2} ( 1 + \begin{matrix}{d}^{2} \\ {a}^{2}\end{matrix} ) - {n}_{1}^{2} ]}^{1 / 2} - {[ 1 + \begin{matrix}{d}^{2} \\ {a}^{2}\end{matrix} ]}^{1 / 2}}$$

where n₁ and n₂ are the refractive indexes of glass and water, d is the thickness of the glass plate.

The drops of treated and non-treated (control water) are closed in a hermetic box with constant temperature, relative humidity and capacity of the air through the box. With a purpose to eliminate the influence of other factors except of investigated the probe and control drops are evaporated simultaneously and the differential energetic spectrum is determined.
Δf(E)=f(probe)−f(control)

The equipment for estimation the biopsychophysical influence of radio, TV and media products on the man, measuring the surface tension and fluctuations of the surface potential of the water drop is realized in a working box with air flux with constant temperature, relative humidity and flux capacity in which a water drop is placed on a pad. The measurement of the surface tension and fluctuations of the surface potential is performed by the help of a nano-brush, which represents five needle's silicon micro-brush, directed with a manipulator by such way that the brush moves parallel to water drops surface. The results of measurement are processed by a computer.

1. Method for estimation the biopsychophysical on the man, characterized in that the person (2) influenced by the product (1) holds in his hands a bottle of water (3), drops from which are placed in a equipment (4), measuring the surface tensions and fluctuations of the surface potential and in a equipment (5), measuring the water intramolecular energy spectrum and the data received together with the data from the equipments (6), measuring the biophysical parameters of the man are processed by a computer (7), connected with data (8) from the previous measurements of the person (2).

2. Equipment for estimation the biopsychophysical influence of radio, TV and media products on the man, characterized in that a drop (4) from the water, influenced via the man by the radio, TV and media products is placed in a hermetic box (4) with an air pump (9) in a plastic folio (5), lying on a glass plate (6) is illuminated through a glass windows (2), with a plane parallel beam from a light source (1), the dark rings received on the bottom surface of the plate are registered by the help of a mirror (10), a digital TV-camera (7) and the water intramolecular energy spectra are calculated with a computer (8).

3. Equipment for estimation the biopsychophysical influence of radio, TV and media products on the man, characterized in that for a water drop (5) placed in the working box (4) with an air pump (7) on a pad (6) and influenced through a hole (1) by a radio, TV and other product, the surface tension and fluctuations of the surface potential are measured by the help of the reader, directed with a manipulator (2), which data is processed by the computer (8).
